(19) United States
(12) Patent Application Publication
(10) Pub. No.: US 2006/0115503 A1
(43) Pub. Date: Jun. 1, 2006

(54) Composition, system and method for treatment of skin

(76) Inventor: Muna C. Goyal, Tarpon Springs, FL (US)

Correspondence Address: MCKEE, VOORHEES & SEASE, P.L.C., 801 GRAND AVENUE, SUITE 3200, DES MOINES, IA, 50309-2721, US

(21) Appl. No.: 10/999,393

(22) Filed: Nov. 30, 2004

(51) Int. Cl. A61K 8/365; A61K 31/19

(52) U.S. Cl. 424/401; 514/557

(57) ABSTRACT

A skin care system and method for treating the signs of aging skin and treating, preventing, and controlling acne is described. The skin care system of the present invention can include combinations of a glycolic cleanser, glycolic scrub mask, glycolic lotion, skin lightening agent, corticosteroid cream, sunscreen and finishing product that work synergistically to improve the healthful appearance of skin or remedy acne.

### I. BACKGROUND OF THE INVENTION

A. Field of Invention

The present invention relates generally to the field of skin care and, more particularly, to topical skin regimens and application systems. Specifically, this invention, in one aspect, relates to a topical system and method for treating the visible signs of aging skin. In another aspect, the invention relates to a topical system and method for treating, preventing, and controlling acne.

B. Problems in the Art

Many persons experience deterioration in skin tone, which happens naturally over time. Some also experience the same as a result of premature aging. Aging skin is characterized by decreased skin renewal. Damage to skin occurs from environmental agents such as sun, wind, and the elements. This can lead to brown spots and wrinkles. Aging skin is also due to oxidant effects from cigarette smoking causing wrinkles, loss of elasticity and decreased tone. This can lead to sagging and inflammation of the skin. Wrinkles, fine lines and skin color irregularity can rob individuals of self-esteem and self-image. Youthful looking skin can increase one's sense of well-being and increase overall health.

Presently, over-the-counter, cosmetic, and pharmaceutical compounds used for treatment of the signs of aging skin give less-than-satisfactory results. No single regimen is known that effectively addresses all the aforementioned concerns. For example, vitamins C and E, elastin, collagen and Co-enzyme Q have shown little clinical effect in this regard. Prescription retinoic acid may help reduce wrinkles, fine lines, and age spots, but is also associated with localized itching, redness, dryness, scaling, irritation, and increased sensitivity to the sun. Therefore, a need has been identified in the art to provide a comprehensive topical skin renewal regimen that interalia, is relatively simple, addresses the multiple concerns of aging skin, and has minimal side effects. Further, available treatments are formulated in incorrect percentages, are too weak, or product application is in the wrong order and timing to provide satisfactory benefit. Therefore, a need has also been identified in the art to provide a novel regimen that incorporates various components in appropriate percentage formulations, and in the most effective order and timing of application to enhance the appearance of aging skin.

Acne is an inflammatory condition of the follicle leading to comedones and pustule formation. Acne is often characterized by excessive oil secretion and can flare due to hormonal imbalance such as seen in teenagers, but is by no means limited to this group. Acne causes psycho-social problems in all age groups, not only in the acute phase, but also because the possibility of chronic scarring exists. Acne scarring is difficult to treat even with the latest laser technologies and peels. Therefore, prevention and control of acne is of paramount importance in avoiding such chronic sequelae. Current conventional treatments address only one component of acne—i.e., inflammation, oil production, or blackhead formation. For example, oral and topical antibiotics are used to decrease bacterial superinfection of the follicular units. However, treatment regimens are often protracted and unsuccessful. Therefore, a need has been identified in the art to avert pustule formation, decrease oil production, and reduce pore size to prevent and control acne. Further, available treatments are formulated in ineffective percentages or are too weak to adequately prevent or resolve body acne. Available treatments have also not provided appropriate order and timing of application to beneficially treat acne skin. Thus, a need has also been identified in the art to provide a novel regimen that has appropriately formulated percentages of the components, and in the most effective order and timing to lead to enhanced appearance of acne skin.

### II. SUMMARY OF THE INVENTION

It is therefore a principal object, feature, advantage, and aspect of the present invention to provide a topical skin care system and method that improves over the state of the art. Further objects, features, advantages, and aspects of the invention include a method and system as described above which:

a. provides a highly effective skin care system and method for treating the visible signs of aging and acne without toxicity.

b. is a simple, effective, and suitable skin care system and method for most mature skin types.

c. provides enhanced and increased youthful appearance of healthy skin.

d. provides a regimen that acts by increasing hyaluronic acid production by increasing exfoliation of dead cells and increasing moisture content of the skin which automatically decreases the appearance of fine lines by decreasing dryness.

e. provides increased brown spot elimination by topical treatment and exfoliation.

f. provides decreased further damage to skin by protection against sun damage.

g. provides decreased pore size by glycolic and astringent methods.

h. provides decreased oil production and inflammation to reduce redness, dryness, and localized pustule formation.

i. provides a completely topical application with minimal side effects.

j. provides a regimen for a variety of skin types, age groups and ethnicity of patients.

These and other objects, features, aspects, and advantages of the present invention will become more apparent with reference to the accompanying specification and claims.

For example, one aspect of the invention includes a general method for topically treating a person's skin using the following materials and steps without regard to order. A two week induction regimen is employed whereby a glycolic cleanser is applied to a person's skin in the morning or in the morning and evening. A glycolic scrub mask is used in the morning or in the evening and then removed from a person's skin. A self heating mask is applied to the person's skin in the evening and then removed. A finishing product and steam penetration are used in the evening. Long-term treatment includes application of a glycolic cleanser and a finishing product in the morning and evening.

Another aspect of the present invention includes a method for topically treating the signs of aging skin whereby a two week induction regimen is instituted. A glycolic cleanser and a skin lightening agent are applied to skin in the morning. Sunscreen can further be applied with the skin lightening agent to protect the skin from further sun damage. A glycolic scrub mask and self-heating mask are used in the evening, followed by steam penetration and a finishing lotion or cold cream. Long-term treatment includes applying a glycolic cleanser in the morning and evening followed by a glycolic cream with or without a sunscreen. The components incorporated into the regimen together are adapted to increase hyaluronic acid production by increasing exfoliation of dead cells and increasing moisture content which results in a decrease of fine lines and an increase in the youthful appearance of skin.

Another aspect of the present invention includes a method for treating acne that employs a two week induction regimen. A glycolic cleanser, glycolic scrub mask and glycolic lotion are applied in the morning. The glycolic cleanser is used again in the evening, followed by a self-heating mud mask, application of cold cream or glycolic lotion, and then steam penetration. Long-term treatment includes applying the glycolic cleanser and glycolic lotion in the morning and evening and then using steam penetration in the evening. The components of the regimen are adapted to work synergistically to decrease pore size, oil production, and pustule formation to control and prevent acne and reduce the possibility of long-term scarring that can result from chronic acne.

Other aspects of the present invention are a system, a collection of compositions, or a kit form that includes a glycolic cleanser, glycolic scrub mask, skin lightening agent, corticosteroid cream, self-heating mask, sunscreen and a finishing product. These components are selected in different combinations and are adapted to work synergistically to treat aging skin and acne.

### III. DETAILED DESCRIPTION OF EXEMPLARY EMBODIMENTS

The present invention will be described as it applies to its preferred embodiment. It is not intended that the present invention be limited to the described embodiment. It is intended that the invention cover all modifications and alternatives which may be included within the spirit and scope of the invention which is defined by the appended claims.

A system according to an exemplary embodiment of the present invention has several components of a regimen including a glycolic cleanser, glycolic scrub mask, skin lightening agent, corticosteroid cream, self-heating mask, sunscreen and a finishing product. The glycolic cleanser is preferably a 9% glycolic acid in a gel or cream base. The glycolic cleanser provides the therapeutic benefit of increased moisturization by the following physiological action of increasing hyaluronic acid production. The glycolic scrub mask is preferably a 1-30% glycolic acid in a honey oatmeal scrub base or a mud mask. It accomplishes these functions by mechanical exfoliation. The glycolic scrub mask provides rapid exfoliation, increased hyaluronic acid production, brown spot removal, and increased circulation of blood in the facial musculature. The glycolic scrub mask further decreases oiliness, decreases bacterial adherence and removes makeup and styling products to increase skin radiance. The skin lightening agent is preferably a 1-6% hydroquinone in a cream or ointment base that provides reduced melasma, brown spots, freckles, pigmentation from acne scarring, and other skin discoloration problems by decreasing melanocyte stimulation. The steroid cream is used as an anti-inflammatory. The topical steroid cream provides decreased inflammation of the skin and temporarily relieves redness, scaling and itching. The topical steroid cream may also prevent or abort acne formation. Further, the topical steroid cream increases the effectiveness of other exfoliative products by decreasing the inflammatory side effects that may result from the exfoliative products. The sunscreen is preferably finely-milled titanium dioxide powder. The sunscreen provides protection from further sun damage to prevent further wrinkles, dryness, brown spots, loss of capillary elastin, and damage from oxidative stress by physical block. The finishing product is a glycolic finishing lotion, glycolic finishing cream, finishing oil or cold cream. The glycolic lotion is preferably a 12% glycolic acid that provides increased moisturization by exfoliation. The glycolic cream is preferably a 12% glycolic acid that provides increased moisturization by exfoliation. The selection of the finishing product is dependent on whether the skin type is adolescent or mature. The finishing product enhances moisture content, suppleness, translucency, and the appearance of hydration by adding moisture. These collection of components are selected in various combinations to improve the visible signs of aging and acne. The components selected are dependent on skin problem, skin type, skin age, skin history, and degree of skin damage. In one aspect of the invention, a collection of components (as chosen or designed for a given functioning person or purpose) is available in kit form.

In another embodiment of the present invention, a preferred method for treating aging skin and acne is described. Several combinations of treatment administration are possible. The method of treatment depends on skin types Fitzpatrick 1-4a, as well as skin conditions, degree of aging, acne, scarring, drying, melasma, brown spots, surface irregularities, etc. The method may be used on any body surface including the face, back, chest, legs, and arms. The following treatment approaches for aging skin and acne are examples offered to illustrate but not limit the invention. Thus, they are presented with the understanding that various formulation modifications may be made and still be within the spirit of the invention.

### EXAMPLE 1

Aging Skin

2 week induction regimen

AM Regimen Skin Age 30-50 Years

Step 1: 9% glycolic gel cleanser

Step 2: 4% hydroquinone cream applied with SPF 15 sunscreen

PM Regimen Skin Age 30-50 Years

Step 1: 12% glycolic scrub prep for 5 minutes in honey oatmeal base, removed with face cloth or rotating facial brush

Step 2: Self-heated mud mask for 5 minute application

Step 3: Steam penetration for 2 minutes

Step 4: Finishing lotion or cold cream

After 2 week induction regimen simplify regimen

AM Regimen Skin Age 30-50 Years

Step 1: 9% glycolic gel cleanser

Step 2: 12% glycolic cream with SPF 15

PM Regimen Skin Age 30-50 Years

Step 1: 9% glycolic gel cleanser

Step 2: 12% glycolic cream

### EXAMPLE 2

Acne

2 week induction regimen

AM Regimen Skin Age 12-90 Years

Step 1: 9% glycolic gel or cream cleanser

Step 2: 12% glycolic scrub mask for 5 minutes

Step 3: 12% glycolic lotion

PM Regimen Skin Age 12-90 Years

Step 1: 9% glycolic gel or cream cleanser

Step 2: Self-heated mud mask for 5 minutes

Step 3: Cold cream or 12% glycolic lotion

Step 4: Steam penetration for 2 minutes

After 2 week induction regimen simplify regimen

AM Regimen Skin Age 12-90 Years

Step 1: 9% glycolic gel or cream cleanser

Step 2: 12% glycolic lotion

PM Regimen Skin Age 12-90 Years

Step 1: 9% glycolic gel or cream cleanser

Step 2: 12% glycolic lotion

Step 3: Steam penetration for 2 minutes

In one experiment, twenty-five patients tried both protocols (as separate components rather than as a regimen as proposed). Improvements were noted at four weeks, 3 months, and 6 months. The regimen disclosed is an improvement over prior known treatments for aging skin because it addresses all the concerns of the signs of aging skin including reducing brown spots, other color irregularity and wrinkles, restoring skin elasticity and tone and improving sagging and inflammation. In addition, the skin care approach is simple, effective for a wide variety of skin types, age and ethnicity and enhances the youthfulness of healthy skin with low toxicity. Moreover, the protocol is completely topical and easy to administer. This promotes an increase in patient compliance, improves patient satisfaction and provides enhanced individual results.

The approach disclosed for acne improves over the prior art by preventing and controlling acne before chronic scarring can occur. The skin care regimen acts as an intervention system to prevent and treat acne by decreasing pore size, oil production and skin inflammation to prevent pustule formation that leads to acne and the possibility of acne scarred skin. The hallmark of this skin care system is that it is simple, can be used in a wide variety of skin types, age, and ethnicity, and is completely topical with minimal side effects.

It will be appreciated that there are additional embodiments which are not illustrated above but which are clearly within the scope and spirit of the present invention. The exemplary embodiment described above is made not by way of limitation to the invention, but for illustration of but one form the invention can take. Variations obvious to those skilled in the art are included within the invention, which is described solely by the claims appended hereto.

What is claimed is:

1. A method for topically treating a person's skin, the method comprising the following steps without regard to order:
(a) applying to the person's skin at a first time and/or at a second time a glycolic cleanser;
(b) applying to the person's skin at or around said first time and/or at or around said second time a glycolic scrub mask;
(c) removing from the person's skin at or around said first time and/or at or around said second time said glycolic scrub mask;
(d) applying to the person's skin at or around said second time a self-heating mask;
(e) applying to the person's skin at or around said second time a steam penetration;
(f) removing from the person's skin at or around said second time said self-heating mask;
(g) applying to the person's skin at or around said first time and/or at or around said second time a finishing product;
(h) repeating steps (a)-(g) periodically.

2. The method of claim 1 further comprising the following steps:
(a) applying to the person's skin at said first and said second time the glycolic cleanser;
(b) applying to the person's skin at or around said first and said second time the finishing product; and
(c) repeating steps (a) and (b) long-term as necessary for the person's skin.

3. The method of claim 1 wherein applying to the person's skin at said first time further comprising application in the morning.

4. The method of claim 1 wherein applying to the person's skin at said second time further comprising application in the evening.

5. The method of claim 1 wherein said repeating steps (a)-(g) periodically further comprising a two week induction period.

6. The method of claim 1 wherein the glycolic cleanser is a 9% glycolic acid composition.

7. The method of claim 6 wherein the 9% glycolic cleanser further comprising said glycolic cleanser in a gel or a cream base.

8. The method of claim 1 wherein the glycolic cleanser provides an increased youthful appearance of skin.

9. The method of claim 1 wherein the glycolic scrub mask is a 1-30% glycolic acid composition.

10. The method of claim 9 wherein the 1-30% glycolic scrub mask further comprising said glycolic scrub mask in a base selected from a honey oatmeal, an almond meal or a mud base.

11. The method of claim 1 wherein the step of applying the glycolic scrub mask further comprising application of said glycolic scrub mask for five minutes.

12. The method of claim 1 wherein the glycolic scrub mask provides an increase in exfoliation of dead skin cells, hyaluronic acid production, and circulation of blood in the facial musculature and facilitates brown spot removal.

13. The method of claim 1 wherein the step of removing from the person's skin the glycolic scrub mask further comprising removal of said glycolic scrub mask with a face cloth or a rotating facial brush.

14. The method of claim 1 wherein the self-heating mask further comprising a mud cream base or a mud powder form.

15. The method of claim 14 wherein the self-heating mask further comprising a particulate matter selected from a oatmeal or an almond meal.

16. The method of claim 15 wherein the self-heating mask further comprising mixing said self-heating mask with water such that the consistency is suitable for application.

17. The method of claim 1 wherein the step of applying the self-heating mask further comprising application of said mask for five minutes.

18. The method of claim 1 wherein the self-heating mask provides a decrease in oiliness and bacterial adherence, and removal of makeup and styling products.

19. The method of claim 1 wherein the step of applying the steam penetration further comprising application of said steam penetration for two minutes.

20. The method of claim 1 wherein the steam penetration provides an increase in moisture content of the person's skin.

21. The method of claim 1 wherein the step of removing from the person's skin the self-heating mask further comprising removal of said self-heating mask with said face cloth or said rotating facial brush.

22. The method of claim 1 wherein the finishing product is selected from a glycolic lotion, a glycolic cream, a finishing oil, or a cold cream.

23. The method of claim 22 wherein the glycolic lotion is a 12% glycolic acid composition.

24. The method of claim 22 wherein the glycolic cream is a 12% glycolic acid composition.

25. The method of claim 1 wherein the finishing product provides an enhanced moisture content, suppleness, translucency, and appearance of hydration of skin.

26. The method of claim 1 wherein topically treating a person's skin further comprising treating the signs of aging skin.

27. The method of claim 26 wherein treating the signs of aging skin further comprising a step of applying to the person's skin at or around said first time a skin lightening agent.

28. The method of claim 27 wherein the skin lightening agent is a 1-6% hydroquinone.

29. The method of claim 28 wherein the 1-6% hydroquinone is in a gel or cream base.

30. The method of claim 29 wherein 1-6% hydroquinone is preferably a 4% hydroquinone cream.

31. The method of claim 27 wherein the skin lightening agent provides an even skin color and a reduction of brown spots.

32. The method of claim 27 wherein the step of applying the skin lightening agent further comprising applying said skin lightening agent with a sunscreen.

33. The method of claim 32 wherein the sunscreen is preferably a finely milled titanium dioxide SPF 15 sunscreen.

34. The method of claim 32 wherein the sunscreen provides a prevention of further damage from the sun.

35. The method of claim 26 wherein the step of applying to aging skin said glycolic cleanser is at said first time.

36. The method of claim 26 wherein the step of applying to aging skin said glycolic cleanser is in a gel base.

37. The method of claim 26 wherein the step of applying to aging skin said glycolic scrub mask is a 12% glycolic acid composition.

38. The method of claim 26 wherein the step of applying said glycolic scrub mask is in said honey-oatmeal base.

39. The method of claim 26 wherein the step of applying said finishing product is said 12% glycolic cream.

40. The method of claim 26 wherein the step of applying at said first and said second time said finishing product long-term is said 12% glycolic cream.

41. The method of claim 40 wherein the step of applying at said first time the 12% glycolic cream further comprising applying said glycolic cream with a sunscreen.

42. The method of claim 41 wherein the sunscreen is preferably a finely milled titanium dioxide SPF 15 sunscreen.

43. The method of claim 41 wherein the sunscreen provides prevention of further damage from the sun.

44. The method of claim 1 wherein topically treating said person's skin further comprising treating, preventing and controlling acne.

45. The method of claim 44 wherein the step of applying to acne skin said glycolic scrub mask is preferably a 12% glycolic acid composition.

46. The method of claim 44 wherein the step of applying said finishing product is preferably said 12% glycolic lotion.

47. The method of claim 44 wherein the step of applying said finishing product long-term is preferably said 12% glycolic lotion.

48. The method of claim 44 wherein said repeating steps (a) and (b) long-term as necessary for the person's skin further comprising a step of applying to the person's skin at or around said second time said steam penetration.

49. A system for topically treating the signs of aging skin comprising:
a glycolic cleanser,
a glycolic scrub mask,
a skin lightening agent,
a corticosteroid cream,
a self-heating mask,
a sunscreen, and
a finishing product.

50. The system of claim 49 wherein the glycolic cleanser is a 9% glycolic acid.

51. The system of claim 50 wherein the 9% glycolic cleanser is in a gel base.

52. The system of claim 49 wherein the glycolic scrub mask is a 12% glycolic acid.

53. The system of claim 52 wherein the 12% glycolic acid is in a honey-oatmeal base.

54. The system of claim 49 wherein the skin lightening agent is a 4% hydroquinone.

55. The system of claim 54 wherein the 4% hydroquinone is in a cream base.

56. The system of claim 49 wherein the corticosteroid cream is a low potency corticosteroid selected from the group consisting of hydrocortisone and desonide.

57. The system of claim 49 wherein the corticosteroid cream is a mid potency corticosteroid selected from a group consisting of triamcinolone and fluocinolone.

58. The system of claim 49 wherein the corticosteroid cream is a high potency corticosteroid selected from a group consisting of clobetasone propionate and halobetasol.

59. The system of claim 49 wherein the self-heating mask further comprising said self-heating mask in a cream base or in a powder base.

60. The system of claim 59 wherein the self-heating mask further comprising a particulate matter selected from a oatmeal or an almond meal.

61. The system of claim 49 wherein the sunscreen is a finely milled titanium dioxide powder SPF 15 sunscreen.

62. The system of claim 49 wherein the finishing product is a glycolic lotion, a glycolic cream, a finishing oil, or a cold cream.

63. The system of claim 62 wherein the glycolic lotion is a 12% glycolic acid composition.

64. The system of claim 62 wherein the glycolic cream is a 12% glycolic acid composition.

65. A system for topically treating, preventing and controlling acne comprising:
a glycolic cleanser,
a glycolic scrub mask,
a corticosteroid cream,
a self-heating mask, and
a finishing product.

66. The system of claim 65 wherein the glycolic cleanser is a 9% glycolic acid.

67. The system of claim 66 wherein the 9% glycolic cleanser is in a gel base or a cream base.

68. The system of claim 65 wherein the glycolic scrub mask is a 12% glycolic acid.

69. The system of claim 68 wherein the 12% glycolic acid is in a base selected from a honey-oatmeal, an almond meal or a mud mask.

70. The system of claim 65 wherein the corticosteroid cream is a low potency corticosteroid selected from the group consisting of hydrocortisone and desonide.

71. The system of claim 65 wherein the corticosteroid cream is a mid potency corticosteroid selected from a group consisting of triamcinolone and fluocinolone.

72. The system of claim 65 wherein the corticosteroid cream is a high potency corticosteroid selected from a group consisting of clobetasone propionate and halobetasol.

73. The system of claim 65 wherein the self-heating mask further comprising said self-heating mask in a cream base or in a powder base.

74. The system of claim 73 wherein the self-heating mask further comprising a particulate matter selected from a oatmeal or an almond meal.

75. The system of claim 65 wherein the finishing product is a glycolic lotion.

76. The system of claim 75 wherein the glycolic lotion is a 12% glycolic acid composition.

77. A collection of compositions for topically treating the signs of aging skin consisting essentially of:
a glycolic cleanser,
a glycolic scrub mask,
a skin lightening agent,
a corticosteroid cream,
a self-heating mask,
a sunscreen, and
a finishing product.

78. The collection of compositions of claim 77 wherein the glycolic cleanser is a 9% glycolic acid.

79. The collection of compositions of claim 78 wherein the 9% glycolic cleanser is in a gel base.

80. The collection of compositions of claim 77 wherein the glycolic scrub mask is a 12% glycolic acid.

81. The collection of compositions of claim 80 wherein the 12% glycolic acid is in a honey-oatmeal base.

82. The collection of compositions of claim 77 wherein the skin lightening agent is a 4% hydroquinone.

83. The collection of compositions of claim 82 wherein the 4% hydroquinone is in a cream base.

84. The collection of compositions of claim 77 wherein the corticosteroid cream is a low potency corticosteroid selected from the group consisting of hydrocortisone and desonide.

85. The collection of compositions of claim 77 wherein the corticosteroid cream is a mid potency corticosteroid selected from a group consisting of triamcinolone and fluocinolone.

86. The collection of compositions of claim 77 wherein the corticosteroid cream is a high potency corticosteroid selected from a group consisting of clobetasone propionate and halobetasol.

87. The collection of compositions of claim 77 wherein the self-heating mask further comprising said self-heating mask in a cream base or in a powder base.

88. The collection of compositions of claim 77 wherein the self-heating mask further comprising a particulate matter selected from a oatmeal or an almond meal.

89. The collection of compositions of claim 77 wherein the sunscreen is a finely milled titanium dioxide powder.

90. The collection of compositions of claim 77 wherein the finishing product is a glycolic lotion, a glycolic cream, a finishing oil, or a cold cream.

91. The collection of compositions of claim 90 wherein the glycolic lotion is a 12% glycolic acid composition.

92. The collection of compositions of claim 90 wherein the glycolic cream is a 12% glycolic acid composition.

93. A collection of compositions for topically treating, preventing and controlling acne consisting essentially of:
a glycolic cleanser,
a glycolic scrub mask,
a corticosteroid cream,
a self-heating mask, and
a finishing product.

94. The collection of compositions of claim 93 wherein the glycolic cleanser is a 9% glycolic acid.

95. The collection of compositions of claim 94 wherein the 9% glycolic cleanser is in a gel or a cream base.

96. The collection of compositions of claim 93 wherein the glycolic scrub mask is a 12% glycolic acid.

97. The collection of compositions of claim 96 wherein the 12% glycolic acid is in a base selected from a honey-oatmeal, an almond meal or a mud mask.

98. The collection of compositions of claim 93 wherein the corticosteroid cream is a low potency corticosteroid selected from the group consisting of hydrocortisone and desonide.

99. The collection of compositions of claim 93 wherein the corticosteroid cream is a mid potency corticosteroid selected from a group consisting of triamcinolone and fluocinolone.

100. The collection of compositions of claim 93 wherein the corticosteroid cream is a high potency corticosteroid selected from a group consisting of clobetasone propionate and halobetasol.

101. The collection of compositions of claim 93 wherein the self-heating mask further comprising said self-heating mask in a cream base or in a powder base.

102. The collection of compositions of claim 101 wherein the self-heating mask further comprising a particulate matter selected from a oatmeal or an almond meal.

103. The collection of compositions of claim 93 wherein the finishing product is a glycolic lotion.

104. The collection of compositions of claim 103 wherein the glycolic lotion is a 12% glycolic acid composition.

105. A kit for topically treating the signs of aging skin comprising:
a glycolic cleanser,
a glycolic scrub mask,
a skin lightening agent,
a corticosteroid cream,
a self-heating mask,
a sunscreen, and
a finishing product.

106. The kit of claim 105 wherein the glycolic cleanser is a 9% glycolic acid.

107. The kit of claim 106 wherein the 9% glycolic cleanser is in a gel base.

108. The kit of claim 105 wherein the glycolic scrub mask is a 12% glycolic acid.

109. The kit of claim 108 wherein the 12% glycolic acid is in a honey-oatmeal base.

110. The kit of claim 105 wherein the skin lightening agent is a 4% hydroquinone.

111. The kit of claim 110 wherein the 4% hydroquinone is in a cream base.

112. The kit of claim 105 wherein the corticosteroid cream is a low potency corticosteroid selected from the group consisting of hydrocortisone and desonide.

113. The kit of claim 105 wherein the corticosteroid cream is a mid potency corticosteroid selected from a group consisting of triamcinolone and fluocinolone.

114. The kit of claim 105 wherein the corticosteroid cream is a high potency corticosteroid selected from a group consisting of clobetasone propionate and halobetasol.

115. The kit of claim 105 wherein the self-heating mask further comprising said self-heating mask in a cream base or in a powder base.

116. The kit of claim 115 wherein the self-heating mask further comprising a particulate matter selected from a oatmeal or an almond meal.

117. The kit of claim 105 wherein the sunscreen is a finely milled titanium dioxide powder.

118. The kit of claim 105 wherein the finishing product is a glycolic lotion, a glycolic cream, a finishing oil or a cold cream.

119. The kit of claim 118 wherein the glycolic lotion is a 12% glycolic acid.

120. The kit of claim 118 wherein the glycolic cream is a 12% glycolic acid.

121. A kit for topically treating, preventing and controlling acne comprising:
a glycolic cleanser,
a glycolic scrub mask,
a corticosteroid cream,
a self-heating mask, and
a finishing product.

122. The kit of claim 121 wherein the glycolic cleanser is a 9% glycolic acid.

123. The kit of claim 122 wherein the 9% glycolic cleanser is in a gel or a cream base.

124. The kit of claim 121 wherein the glycolic scrub mask is a 12% glycolic acid.

125. The kit of claim 124 wherein the 12% glycolic acid is in a base selected from a honey-oatmeal, an almond meal or a mud mask.

126. The kit of claim 121 wherein the corticosteroid cream is a low potency corticosteroid selected from the group consisting of hydrocortisone and desonide.

127. The kit of claim 121 wherein the corticosteroid cream is a mid potency corticosteroid selected from a group consisting of triamcinolone and fluocinolone.

128. The kit of claim 121 wherein the corticosteroid cream is a high potency corticosteroid selected from a group consisting of clobetasone propionate and halobetasol.

129. The kit of claim 121 wherein the self-heating mask further comprising said self-heating mask in a cream base or in a powder base.

130. The kit of claim 121 wherein the self-heating mask further comprising a particulate matter selected from a oatmeal or an almond meal.

131. The kit of claim 130 wherein the finishing product is a glycolic lotion.

132. The kit of claim 130 wherein the glycolic lotion is a 12% glycolic acid.
